(19) United States
(12) Patent Application Publication
(10) Pub. No.: US 2016/0166985 A1
(43) Pub. Date: Jun. 16, 2016

(54) PHOTOBIOREACTOR SYSTEM FOR AIR PURIFICATION BY USING MICROALGAE

(71) Applicant: Nano and Advanced Materials Institute Limited, Hong Kong (HK)

(72) Inventors: YEUK TIN LAU, Hong Kong (HK); Lok Hang Roger KEUNG, Hong Kong (HK); Sik Chun Johnny LO, Hong Kong (HK); Jonathan Christianto, Hong Kong (HK)

(21) Appl. No.: 14/968,931

(22) Filed: Dec. 15, 2015

Related U.S. Application Data
(60) Provisional application No. 62/124,348, filed on Dec. 16, 2014.

(51) Int. Cl. B01D 53/84; C12M 1/36; C12M 1/34; C12M 1/00

(52) U.S. Cl. CPC B01D 53/84; C12M 21/02; C12M 29/06; C12M 41/48; C12M 41/34; C12M 41/40; C12M 41/26

(57) ABSTRACT

The present invention relates to air purification. The present invention provides a system for reducing carbon dioxide concentration in air in locations with sub-tropical to temperate climates and method of reducing carbon dioxide concentration using the system.

### CROSS REFERENCE TO RELATED APPLICATION

Pursuant to 35 U.S.C. §119(e), this is a non-provisional patent application claiming benefit from U.S. provisional patent application Ser. No. 62/124,348 filed Dec. 16, 2014 and titled “Photobioreactor design for indoor and outdoor air purifications in Hong Kong using microalgae”, and the disclosure of which is incorporated herein by reference in its entirety.

### FIELD OF THE INVENTION

The present invention relates to air purification. In particular, the present invention relates to a system for reducing carbon dioxide concentration in air using freshwater or marine photosynthetic microalgae. The present invention is particularly suitable for use in places of sub-tropical and temperate climates, with average temperature between 15-30° C.

### BACKGROUND

Emission of greenhouse gases from vehicles and industries is one of the major causative factors for global warming and leads to temperature increase on Earth. Carbon dioxide (CO₂) accounts for about 68% of the greenhouse gas emissions from human activities. CO₂ sequestration by various methods has been widely studied, which aims to reduce the emission of CO₂ to the atmosphere. Conventionally, CO₂ in air is captured by chemical and physical technologies, such as scrubbing with aqueous amine solutions together with solvent regeneration at high temperature and physical solvent separation using high pressure. The captured CO₂ is then transported to offshore geological storage (Gibbins et al., 2008).

Biological method for air purification is now being considered as a better alternative as compared to the conventional chemical and physical methods. Biological method requires milder conditions. High temperature and pressure, and environmental harmful solvents used in conventional methods are excluded in biological method. Biological method is also more sustainable. Biological method relies on photosynthesis to convert unwanted CO₂ to oxygen, thus physical storage space for storing CO₂ is not needed. Additional to oxygen, valuable biomass which can be used for biodiesel production, fish feed, fertilizer and chemicals which are used for health care and food products are produced during removal of CO₂ by biological methods (Chisti, 2007).

However, local climate affects performance of the photobioreactor in removing carbon dioxide. Therefore, it is desired to provide a photobioreactor system which is adapted to the local climate and environment factors for efficient removal of carbon dioxide in air.

### SUMMARY OF THE INVENTION

Among photosynthetic biomass organisms, freshwater microalgae are regarded to be the most suitable photosynthetic organisms for the application of air purification photobioreactor system, due to its high photosynthetic efficiency (Perrin et al., 2012).

Accordingly, the present invention provides a system for air purification specific for locations with sub-tropical to temperate climate using microalgae.

A first aspect of the present invention provides a photobioreactor system for reducing carbon dioxide in air in a location with sub-tropical to temperate climate comprises:

a reactor tank for housing a microalgae culture medium comprising Chlorella species at an initial concentration of 1,000,000-1,500,000 cells/ml at a temperature of 15-30° C.;

a gas inlet where the air enters into the system;

a gas pump, a gas pressure controller and a sparger for feeding the air to the microalgae culture medium at a flow rate of 0.1-20 L/min as small bubbles;

a temperature controller to regulate the temperature of the microalgae culture medium to the desired temperature for the operation;

a light source for providing light to the microalgae culture medium with an intensity of 50-500 μmolm⁻²s⁻¹PPFD; and

a gas outlet where purified gas having a reduced carbon dioxide concentration exits the system.

In accordance with an embodiment of the presently claimed invention, the location with sub-tropical to temperate climate is Southeast Asia.

In accordance with another embodiment of the presently claimed invention, the location is Hong Kong.

In accordance with yet another embodiment of the presently claimed invention, the temperature of the microalgae culture medium is controlled at 15-30° C.

In accordance with another embodiment of the presently claimed invention, the culture medium is maintained at pH 7-9.

In accordance with another embodiment of the presently claimed invention, said light source comprises light emitting diode, fluorescence tube, sunlight or all.

A second aspect of the presently claimed invention is to provide a method for reducing carbon dioxide in air in a location with sub-tropical to temperate climate comprises:
providing a photobioreactor system;
feeding the air being treated into the photobioreactor system via a gas inlet;
regulating the temperature of the microalgae culture medium to the desired temperature for the operation;
feeding the air being treated to the microalgae culture medium at a flow rate of 0.1 to 20 L/min as small bubbles.

Unlike any existing photobioreactor, the present photobioreactor system and method of air purification are adapted to sub-tropical to temperate climates for high efficient removal of carbon dioxide.

### BRIEF DESCRIPTION OF THE DRAWINGS

Embodiments of the present invention are described in more detail hereinafter with reference to the drawings, in which:

FIG. 1 is a schematic diagram of a panel shaped photobioreactor of the present invention, for indoor air purification applications;

FIG. 2 is a schematic diagram of a tubular shaped photobioreactor of the present invention, for indoor air purification applications.

FIG. 3 is a schematic diagram of photobioreactor system with six tubular shaped reactors of the present invention, for outdoor air purification applications.

FIG. 4 is a graph showing CO₂ consumption at different time in Example 1.

FIG. 5 is a graph showing CO₂ consumption at different time in Example 2.

FIG. 6 is a graph showing CO₂ consumption at different time in Example 3.

### DETAILED DESCRIPTION OF THE INVENTION

To illustrate the structure and advantages of the present invention, below is the detailed description of the present invention in combination with the figures and embodiments.

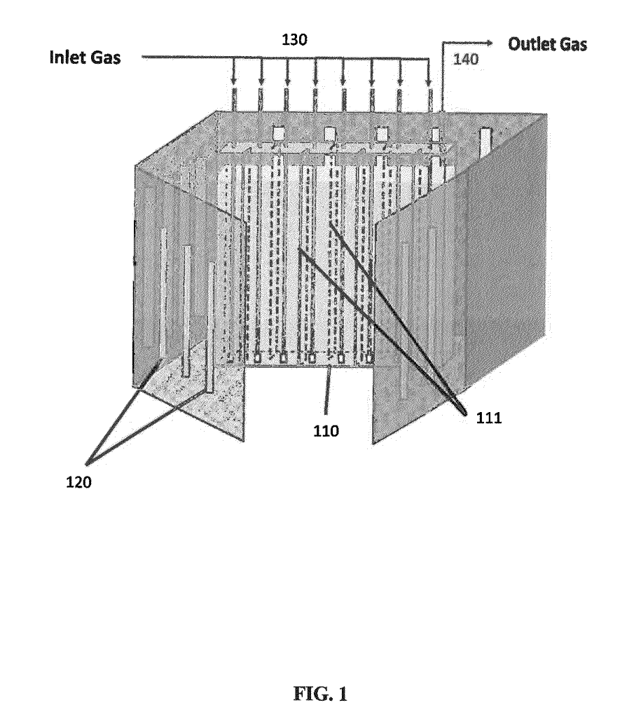

As shown in FIG. 1, the photobioreactor system of the present invention comprises a flat panel shaped reactor tank 110 for housing microalgae culture medium. The reactor tank of the present invention may be transparent, translucent or reflective such that light can pass through the reactor tank and reaches to the microalgae housed therein for photosynthesis. The material of the reactor tank of the present invention may be glass, acrylic, polypropene, or any other transparent material used in the art. The microalgae culture medium comprises Chlorella species at an initial concentration of 1,000,000-1,500,000 cells/ml. Chlorella species applicable for the present invention includes, but are not limited to, Chlorella sp., Chlorella pyrenoidosa, Chlorella vulgaris, etc. The reactor tank may or may not be partitioned by a plurality of partitions 111 to arrange various internal flow patterns. For instance, the plurality of partitions 111 consist of partitions projecting from opposite sides of the reactor tank alternatively. In one embodiment, the reactor tank does not have any partitions. In another embodiment, the reactor tank has two equally separate partitions to create an airlift flow pattern. The photobioreactor system of the present invention further comprises one or more gas inlet 130 for feeding the air being treated into the microalgae culture medium. The air being treated is fed into the microalgae culture medium as bubbles by sparger or any other applicable means readily used in the art. In one embodiment, a gas inlet extended from the top to the bottom of the reactor tank feed in air into the microalgae culture medium at a flow rate of 0.1-2.0 L/min. In another embodiment, air being treated is directly bubbled into the microalgae culture medium through gas inlet at the bottom of the reactor tank at a flow rate of 0.1-2.0 L/min. The temperature of the microalgae culture medium is controlled at 15-30° C. A water bath or any other temperature control system in the art may be used in the present system to control the temperature of the microalgae culture medium. In one embodiment, the temperature of the microalgae culture medium is being maintained at 15-30° C. In another embodiment, the temperature of the microalgae culture medium and the air being treated are the same. The present photobioreactor system includes a temperature controller (not shown in FIG. 1) which, regulate the temperature of the microalgae culture medium to the desired temperature for the operation. The medium for culturing the microalgae Chlorella species may be any type of medium suitable for growth of the microalgae. A mixture of medium may also be used. In one embodiment, the medium is Bold's basal medium. In another embodiment, the medium can be Bristol's medium. pH of the culture medium is also maintained at pH 7-9. When the present photobioreactor is operating continuously, density of the microalgae culture medium and pH may be maintained at 1,000,000-1,500,000 cells/ml by removing the microalgal biomass and replacing fresh culture medium in the reactor tank. In one embodiment, the operation cycle of the microalgae photobioreactor is two weeks. After continuous operation for two weeks. The density of the culture medium is diluted back to the initial concentration of 1,000,000-1,500,000 cells/ml, and fresh culture medium is added back into the reactor tank. Algae biomass can be optionally harvested from the microalgae culture medium.

The photobioreactor system further comprises a gas outlet 140 where purified air with reduced carbon dioxide exits the reactor tank. In one embodiment, the gas outlet is positioned at upper portion of the photobioreactor. The gas being treated are bubbled into the microalgae culture medium at the bottom of the reactor tank and are purified by the microalgae culture such that purified air with reduced carbon dioxide rises to the top of the reactor tank and exits through the gas outlet. The Chlorella species of the microalgae culture medium carries out photosynthesis consuming carbon dioxide in the air being treated. The microalgae culture medium converts carbon dioxide into oxygen. Thus, the air after treatment with the microalgae culture medium is purified with reduced carbon dioxide.

The photobioreactor system of the present invention further comprises a light source for providing light to the microalgae culture medium for photosynthesis. In one embodiment, the light source is in a form of a light chamber 120 of array of LEI) lights situated around the outside of the reactor tank 110. Fluorescent lamps, high-pressure sodium map, fluorescent mercury lamp, sunlamp, or sunlight may be used. In one embodiment, a light intensity of 50-500 μmolm⁻²s⁻¹PPFD is supplied to the microalgae culture medium for efficient removal of carbon dioxide. In an exemplary embodiment, the array of LED lights or other light source may be situated in a position perpendicular to the orientation of the plurality of the partitions 111 such that the photosynthetic rate can be increased by maximizing the light source reaching to the microalgae culture medium inside the reactor tank 110.

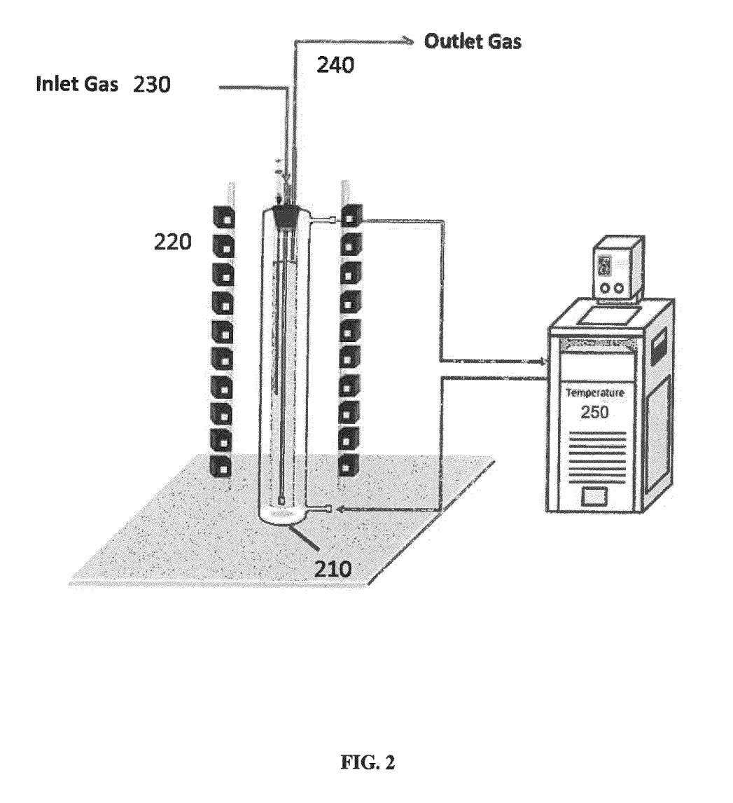

FIG. 2 illustrates another embodiment of the present photobioreactor in which reactor tank 210 is tubular in shape. The diameter to height ratio of the reactor tank may be between 1:5 to 1:10. A vertically arranged array of LED lights 220 positioned along the tubular reactor tank, a gas inlet 230 extended to the bottom of the reactor tank to feed air into the microalgae culture medium and a gas outlet 240 at the top of the reactor tank are shown. The photobioreactor system further comprises sensors to monitor temperature, gas flow rate, light intensity and pH in the reactor tank and corresponding control means to maintain the desirable ranges of temperature, light intensity, flow rate and pH for efficient carbon dioxide removal in sub-tropical to temperate climate. In this embodiment, a temperature controller 250 is used to monitor the temperature of the microalgae culture medium and adjust the temperature when it is different for the desired operation temperature.

The presently claimed invention also provides a method for reducing carbon dioxide in air in a location with sub-tropical to temperate climate comprises providing a photobioreactor system in accordance with the present invention, which comprises of, feeding the air into the bioreactor system, temperature controller system. The method of reducing carbon dioxide for purification of the present invention comprises maintaining the microalgae culture medium at 1,000,000-1,500,000 cells/mL at 15-30° C., adjusting the temperature of the microalgae culture medium to be the same as the desired operation temperature, feeding the incoming gas being treated into the microalgae culture medium at 0.1 to 2.0 L/min, enabling purified gas having reduced concentration of carbon dioxide than the incoming gas to exit the photobioreactor system and optionally collecting any biomass produced during photosynthesis of the microalgae culture medium.

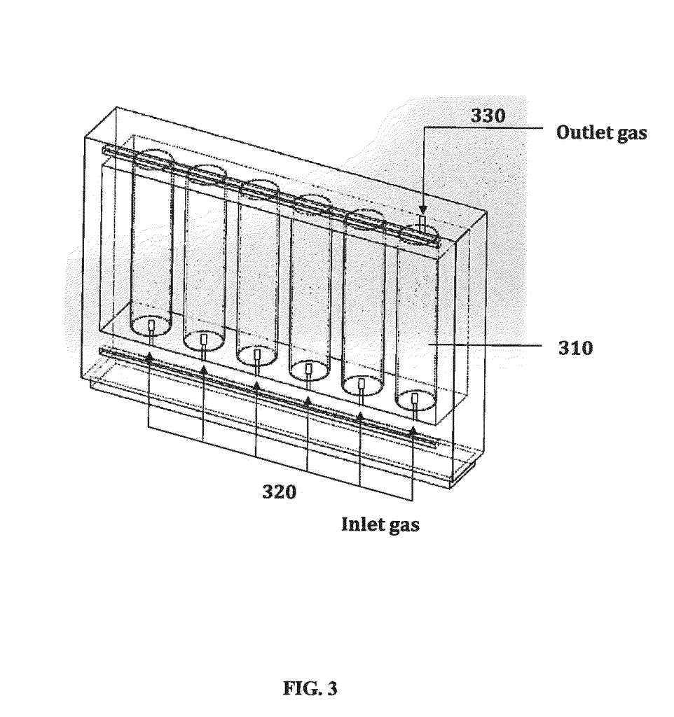

FIG. 3 illustrates another embodiment of the present photobioreactor in which reactor tank 310 is tubular in shape and is used for outdoor air purification application. The diameter to height ratio of the reactor tank may be between 1:5 to 1:10. The material of the reactor tank of the present invention may be glass, acrylic, polypropene, or any other transparent material used in the art. In one embodiment, a series of six tubular reactor tanks are installed in vertical alignment for the air purification. A gas inlet 320 is installed in the bottom of each reactor tank to feed air into the microalgae culture medium and multiple spargers or any gas dispersion device readily used in the art is used to evenly disperse air being treated throughout the reactor tank. A gas outlet 330 is installed in the top portion of the reactor tank. The photobioreactor system further comprises sensors to monitor temperature, humidity, gas flow rate, light intensity, and CO₂ inlet and outlet concentration in the reactor tank and corresponding control means to maintain the desirable ranges of flow rate for efficient carbon dioxide removal in sub-tropical to temperate climate. In this embodiment, natural sunlight is used for the source of illumination and temperature of the system equals that of the environmental temperature in sub-tropical to temperate climate.

The presently claimed invention also provides a method for reducing carbon dioxide in air in a location with sub-tropical to temperate climate comprises providing a photobioreactor system in accordance with the present invention, which comprises of, feeding the air into the bioreactor system, dispersing the air evenly into the bioreactor system. The method of reducing carbon dioxide for purification of the present invention comprises maintaining the microalgae culture medium at 1,000,000-1,500,000 cells/mL, feeding the incoming gas being treated into the microalgae culture medium at 1 to 20 L/min, enabling purified gas having reduced concentration of carbon dioxide than the incoming gas to exit the photobioreactor system and optionally collecting any biomass produced during photosynthesis of the microalgae culture medium.

The present photobioreactor system demonstrates highly efficient carbon dioxide purification in locations with sub-tropical to temperate climate. The microalgae species, culture density, temperature, flow rate and light intensity are selected for high efficiency carbon dioxide purification in locations with sub-tropical to temperate climates. The locations have hot, humid summers and generally mild winters. In particular, the location has a mean monthly temperature between 15° C. and 30° C. The present photobioreactor system is designed for high efficiency carbon dioxide purification in Southeast Asia. The present photobioreactor system is designed for high efficiency carbon dioxide purification in Hong Kong or location with similar climate.

Three examples are given below to demonstrate the operation of the present invention for air purification in Hong Kong using microalgae which can significantly reduce the CO₂ concentration in air.

### Example 1

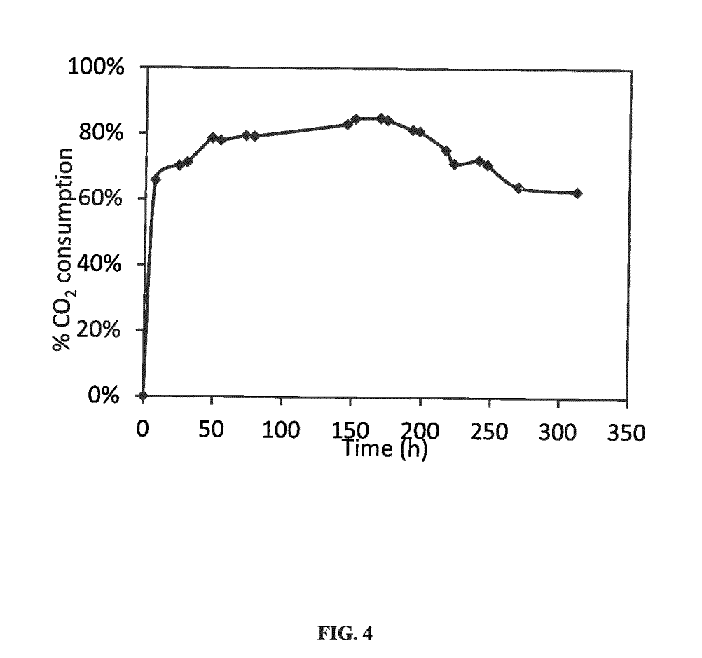

Purification of CO₂ polluted air using the present invention as shown in FIG. 1 is carried out. The reactor is panel shaped reactor with a single partition. The volume of the reactor is 4 L. The reactor is operated indoors. An initial concentration of Chlorella sp. in modified Bold's Basal medium is 1,200,000 cells of microalgae/mL medium. The temperature of the microalgae culture medium is 30° C. A LED setup radiates light continuously at 400 μmol/m²s⁻¹. The operation cycle is two weeks. The CO₂ concentration of the inlet gas is 490 ppm and its flow rate is 1000 mL/min. After 24 hours, the concentration of the CO₂ in the outlet gas is measured to be 146 ppm, with a consumption of 70% CO₂. The reactor is able maintain over 70% CO₂ consumption for the two week operation cycle. The graph showing the CO₂ consumption performance over the operation period of two weeks is shown in FIG. 4

### Example 2

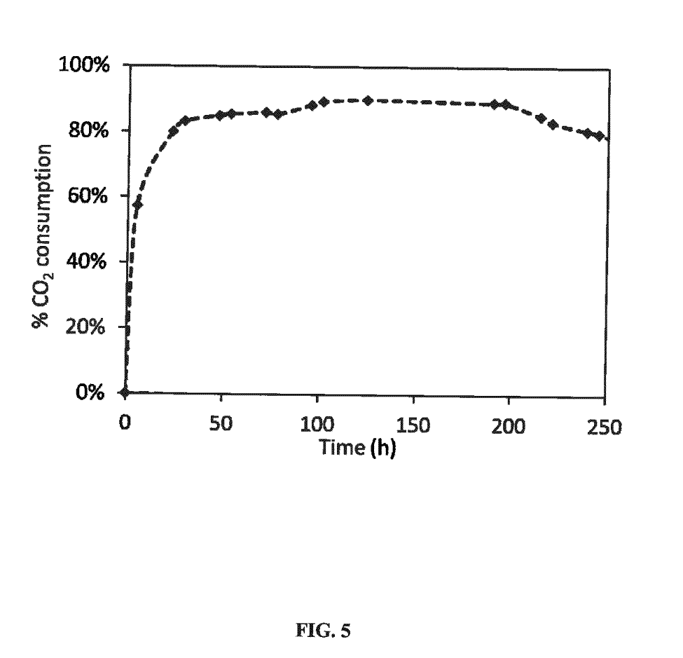

Purification of CO₂ polluted air using the present invention as shown in FIG. 2 is carried out. The reactor is a tubular reactor with a volume of 1000 ml, the diameter to height ratio is 1:10. The reactor is operated indoors. An initial concentration of Chlorella sp. in modified Bold's Basal medium is 1,200,000 cells of microalgae/mL medium. The temperature of the microalgae culture medium is 30° C. A LED setup radiates light continuously at 400 μmol/m²s⁻¹. The CO₂ concentration of the inlet gas is 450 ppm and its flow rate is 1000 mL/min. After 24 hours, the concentration of the CO₂ in the outlet gas is measured to be 90 ppm, with a CO₂ consumption of over 80% The reactor is able maintain over 80% CO₂ consumption for 250 hours of operation. The graph showing the CO₂ consumption performance over the operation period of two weeks is shown in FIG. 5.

### Example 3

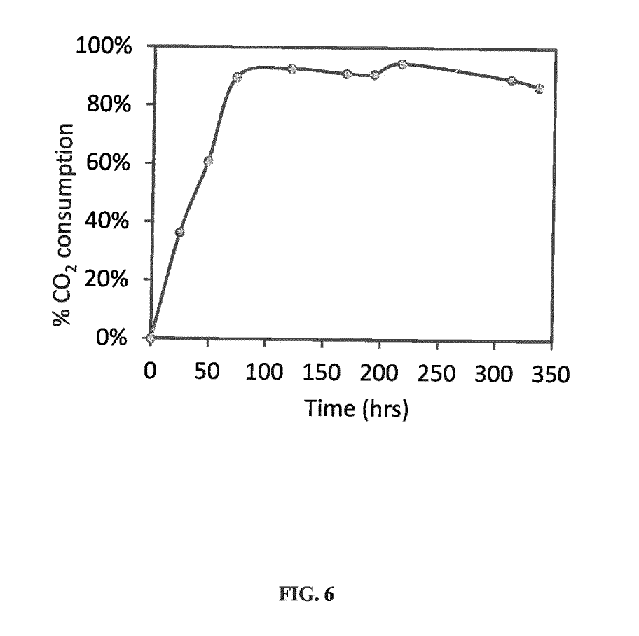

Purification of CO₂ polluted air using the present invention as shown in FIG. 3 is carried out. The reactor is a tubular reactor with a volume of 100 L, the diameter to height ratio is 1:5. Six reactors are operating in parallel. The reactor is operated in an outdoor site. An initial concentration of Chlorella sp. in modified Bold's Basal medium is 1,200,000 cells of microalgae/mL medium. Sunlight provides natural illumination to the microalgae culture medium. The CO₂ concentration of the inlet gas is 400 ppm and its flow rate is 10 L/min. After 72 hours, the concentration of the CO₂ in the outlet gas is measured to be 45 ppm, with a CO₂ consumption of over 80% The reactor is able maintain over 80% CO₂ consumption for 18 days of operation and 40%. CO₂ consumption for 30 days of operation. The graph showing the CO₂ consumption performance over the operation period of one month is shown in FIG. 6

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated. It is intended that the scope of the invention be defined by the following claims and their equivalence.

### Appendix 1

### Bristol Medium:

### Bristol A

| Chemical Name | Stock | |
|---|---|---|
| Sodium Nitrate (NaNO₃) | 20-25 | g/L |
| Dipotassium Phosphate (K₂HPO₄) | 6-7.5 | g/L |
| Monopotassium Phosphate (KH₂PO₄) | 14-17.5 | g/L |
| Sodium Chloride (NaCl) | 2-2.5 | g/L |

### Bristol B

| Chemical Name | Stock |
|---|---|
| Calcium Chloride (CaCl₂) | 1.5-2 g/L |

### Bristol C

| Chemical Name | Stock |
|---|---|
| Magnesium Sulfate Heptahydrate (MgSO₄•7H₂0) | 6-7.5 g/L |

Bristol A, Bristol B, Bristol C solution is diluted 100 fold to working volume

### Trace Medium:

### Trace 1 Alkaline EDTA

| Chemical Name | Stock |
|---|---|
| Etylenediaminetetraacetic acid (EDTA) | 40-50 g/L |
| Potassium hydroxide (KOH) | 25-31 g/L |

### Trace 2 Trace Metal

| Chemical Name | Stock |
|---|---|
| Zinc Sulfate Heptahydrate (ZnSO₄•7H₂0) | 7.05-8.82 g/L |
| Manganese(II) Chloride Tetrahydrate (MnCl₂•4H₂O) | 1.15-1.44 g/L |
| Sodium Molybdate (Na₂MoO₄) | 0.95-1.19 g/L |
| Copper(II) Sulfate Pentahydrate (CuSO₄•5H₂O) | 1.25-1.57 g/L |
| Cobalt(II) Chloride Hexahydrate (CoCl₂•6H₂O) | 0.32-0.40 g/L |

### Trace 3 Acidified Acid

| Chemical Name | Stock | |
|---|---|---|
| Iron(II) Sulfate Heptahydrate (FeSO₄•7H₂O) | 3.98-4.98 | g/L |
| Sulfuric Acid (H₂SO₄) | 0.8-1 | mL/L |

### Trace 4

| Chemical Name | Stock |
|---|---|
| Boric Acid (H₃BO₃) | 9.14-11.42 g/L |

Trace 1, Trace 2, Trace 3, Trace 4 solution is diluted 1000 fold, to working volume

1. A photobioreator system for reducing carbon dioxide in air in a location with sub-tropical to temperate climate comprising:
a reactor tank configured to house a microalgae culture medium comprising Chlorella species;
a gas inlet configured to pump air into the system;
a gas pump configured to control a flow rate of the air;
a gas pressure controller configured to control a gas pressure of the air;
a sparger configured to feed the air to the microalgae culture medium as small bubbles;
a temperature controller configured to sense and regulate a temperature of the microalgae culture medium to a desired temperature;
a plurality of CO₂ concentration sensors configured to monitor CO₂ concentration;
a light controller configured to sense and control a light intensity;
a pH meter configured to monitor a pH value of the culture medium;
a relative humidity sensor configured to monitor a relative humidity of the air; and
a gas outlet configured to discharge purified gas having a reduced carbon dioxide concentration exited in the system.

2. The system of claim 1, wherein the location is Southeast Asia or Hong Kong.

3. The system of claim 1, wherein the Chlorella species are selected from a group consisting of Chlorella sp., Chlorella pyrenoidosa, Chlorella vulgaris and combination thereof.

4. The system of claim 1, wherein the culture medium is Bristol's medium.

5. The system of claim 1, wherein a shape of the reactor tank is in flat panel.

6. The system of claim 5, wherein the reactor tank is partitioned by a plurality of partitions configured to arrange a plurality of internal flow patterns.

7. The system of claim 1, wherein a shape of the reactor tank is in tubular.

8. The system of claim 7, wherein a diameter to height ratio of the reactor tank ranges from 1:5 to 1:10.

9. The system of claim 7, wherein a plurality of reactor tanks is installed in a vertical alignment.

10. The system of claim 1, wherein the plurality of CO₂ concentration sensors comprise at least one CO₂ inlet concentration sensor and at least one CO₂ outlet concentration sensor.

11. The system of claim 1, wherein the pH value of the culture medium ranges from 7 to 9.

12. The system of claim 1, wherein a material of the reactor tank of the present invention is selected from a group consisting of glass, acrylic and polypropene.

13. The system of claim 1, wherein the flow rate of the air ranges from 0.1 L/min to 20 L/min.

14. The system of claim 1, wherein the temperature of the microalgae culture medium ranges from 15° C. to 30° C.

15. The system of claim 1, wherein the light intensity ranges from 50 μmolm⁻²s⁻¹PPFD to 500 μmolm⁻²s⁻¹PPFD.

16. A method for reducing carbon dioxide in air by using the system of claim 1 in a location with sub-tropical to temperate climate, the method comprises:
feeding air into the system via the gas inlet;
adjusting a temperature of the microalgae culture medium to a desired operation temperature;
providing light to the microalgae culture medium at a predetermined intensity;
feeding the air to the microalgae culture medium at a flow rate as small bubbles; and
obtaining a purified gas from a gas outlet;
diluting back a density of the microalgae culture medium to an initial concentration at a preset interval.

17. The method of claim 16, further comprising: collecting biomass produced during photosynthesis.

18. The method of claim 16, wherein the location is Southeast Asia or Hong Kong.

19. The method of claim 16, wherein the Chlorella species is selected from a group consisting of Chlorella sp., Chlorella pyrenoidosa, Chlorella vulgaris and combinations thereof.

20. The method of claim 16, wherein the culture medium is Bristol's medium.

21. The method of claim 16, wherein the preset interval is two weeks.
